(19) United States
(12) Patent Application Publication
(10) Pub. No.: US 2015/0084641 A1
(43) Pub. Date: Mar. 26, 2015

(54) CONTROL DEVICE AND TEST METHOD

(71) Applicant: SEIKO EPSON CORPORATION, Tokyo (JP)

(72) Inventors: Daisuke KAWAKAMI, Matsumoto-shi (JP); Shinichi MIYAZAKI, Suwa-shi (JP); Tsunemori ASAHI, Azumino-shi (JP)

(21) Appl. No.: 14/494,347

(22) Filed: Sep. 23, 2014

(30) Foreign Application Priority Data
Sep. 24, 2013 (JP) 2013-196842

(51) Int. Cl. G01R 31/02; A61B 17/3203; G01M 99/00

(52) U.S. Cl. CPC G01R 31/02; G01M 99/005; A61B 17/3203; A61B 2017/00725
USPC 324/511

(57) ABSTRACT

A control device is connectable to a surgical instrument, and controls the surgical instrument. When the surgical instrument is connected, the control device determines the quality of the surgical instrument by performing a plurality of different tests.

This patent application claims the benefit of Japanese Patent Application No. 2013-196842, filed on Sep. 24, 2013. The content of the aforementioned application is hereby incorporated by reference in its entirety.

### BACKGROUND

1. Technical Field

The present invention relates to a test.

2. Related Art

An ultrasonic probe used for ultrasonic diagnosis may need to be cleaned and disinfected after being used once. A technique of determining whether or not cleaning and disinfection of an ultrasonic probe have already completed using an RFID stored in the ultrasonic probe and notifying a user of the result before the user uses the ultrasonic probe is known (for example, JP-A-2010-282358).

The problem of the aforementioned related art is that the test of the electrical system of medical equipment has not been taken into consideration. In addition, miniaturization of a device, low cost, resource saving, ease of manufacture, improvement in usability, and the like have been demanded.

### SUMMARY

An advantage of some aspects of the invention is to solve at least one of the problems described above, and the invention can be implemented as the following forms.

(1) An aspect of the invention provides a control device that is connectable to a surgical instrument and outputs a control signal for controlling the surgical instrument. When the surgical instrument is connected, the control device determines the quality of the surgical instrument by performing a plurality of different tests. According to the aspect, it is possible to test the quality of the surgical instrument, for example, the quality of an electrical system.

(2) In the control device of the aspect described above, the surgical instrument may have an identifier, the control device may includes a test unit that applies a voltage to the surgical instrument and an identification unit that identifies the identifier, and the test unit may change the voltage according to the identifier. According to this configuration, a test according to an identifier can be performed.

(3) In the control device of the aspect described above, the surgical instrument may have an identifier, the control device may include a first test unit that applies a first voltage to the surgical instrument, an identification unit that identifies the identifier, and a second test unit that applies a second voltage higher than the first voltage to the surgical instrument, and the second test unit may operate after operations of the first test unit and the identification unit. According to this configuration, since a first test using the first voltage lower than the second voltage is performed before a second test, the second test can be omitted when an abnormality is detected in the first test.

(4) Another aspect of the invention provides a control device that is connectable to a liquid ejection mechanism having an identifier and outputs a control signal for controlling the liquid ejection mechanism. The control device includes: a first test unit that applies a first voltage to the liquid ejection mechanism; an identification unit that identifies the identifier; and a second test unit that applies a second voltage higher than the first voltage to the liquid ejection mechanism. The second test unit operates after operations of the first test unit and the identification unit. According to this aspect, since a first test using the first voltage lower than the second voltage is performed before a second test, the second test can be omitted when an abnormality is detected in the first test.

(5) In the control device described above, the liquid ejection mechanism may be connectable, and the identification unit may operate when the liquid ejection mechanism is connected. According to this configuration, as a part of the preparation for using the surgical instrument, the user can connect the surgical instrument to the control device so that the control device can identify the identifier.

(6) Still another aspect of the invention provides a test method of medical equipment including a surgical instrument having an identifier and a control device that outputs a driving signal to the surgical instrument. The test method includes: applying a first voltage to the surgical instrument; identifying the identifier; and applying a second voltage higher than the first voltage to the surgical instrument. The application of the second voltage is performed after the application of the first voltage and the identification of the identifier. According to this aspect, since the first and second voltages are applied, it is possible to perform a test that is not possible only by acquiring the identifier. Since the application of the first voltage using the first voltage lower than the second voltage is performed before the application of the second voltage, the application of the second voltage can be omitted when an abnormality is detected in the application of the first voltage. The surgical instrument can be tested by combining a test performed after acquiring the identifier and a test that is not relevant to the presence of an identifier.

(7) In the test method of the aspect described above, the identifier may have different information for each type of the surgical instrument. According to this configuration, the type of the surgical instrument can be identified by the identifier. As a result, the type of the surgical instrument can be reflected in the application of the second voltage.

(8) In the test method of the aspect described above, a value of the second voltage may be changed according to the identifier. According to this configuration, the second voltage can be changed according to the type of the surgical instrument.

(9) In the test method of the aspect described above, a value of the second voltage may be five times or more than a value of the first voltage.

(10) In the test method of the aspect described above, the identification of the identifier may be performed before the application of the first voltage. According to this configuration, the application of the first voltage and the application of the second voltage can be performed after the acquisition of the identifier.

(11) In the test method of the aspect described above, the application of the first voltage may be performed before the identification of the identifier. According to this configuration, the identifier can be acquired after the application of the first voltage.

(12) In the test method of the aspect described above, the identifier may have information unique to each surgical instrument. According to this configuration, each of the surgical instruments can be identified by the identifier.

(13) In the test method of the aspect described above, when an identifier acquired in the identification of the identifier is the same as an identifier acquired in the previous identification of the identifier, the application of the second voltage may not be performed. According to this configuration, it is possible to prevent the surgical instrument from being reused.

(14) In the test method of the aspect described above, the surgical instrument may include an actuator. According to this configuration, it is possible to perform a test associated with the actuator.

(15) In the test method of the aspect described above, the medical equipment may be a liquid ejection mechanism. According to this configuration, it is possible to perform a test for the liquid ejection mechanism.

The invention can also be implemented in various forms other than the above. For example, the invention can be implemented in forms such as a program for implementing a test method, a storage medium in which the program is stored, and a control device for carrying out a test method. Alternatively, the invention can be implemented in a form, such as a liquid ejection mechanism or medical equipment including the above-described control device.

### BRIEF DESCRIPTION OF THE DRAWINGS

The invention will be described with reference to the accompanying drawings, wherein like numbers reference like elements.

FIG. 1 is a configuration diagram of a liquid ejection apparatus.

FIG. 2 is a block diagram showing the internal configuration of a control device.

FIG. 3 is a flowchart showing the first half of the test process.

FIG. 4 is a flowchart showing the second half of the test process.

FIGS. 5A to 5E are graphs showing various waveforms in the short circuit test.

FIGS. 6A to 6C are graphs showing various waveforms in the disconnection test.

FIGS. 7A to 7C are graphs showing various waveforms in the overcurrent test.

FIGS. 8A to 8C are graphs showing various waveforms in the insulation test.

### DESCRIPTION OF EXEMPLARY EMBODIMENTS

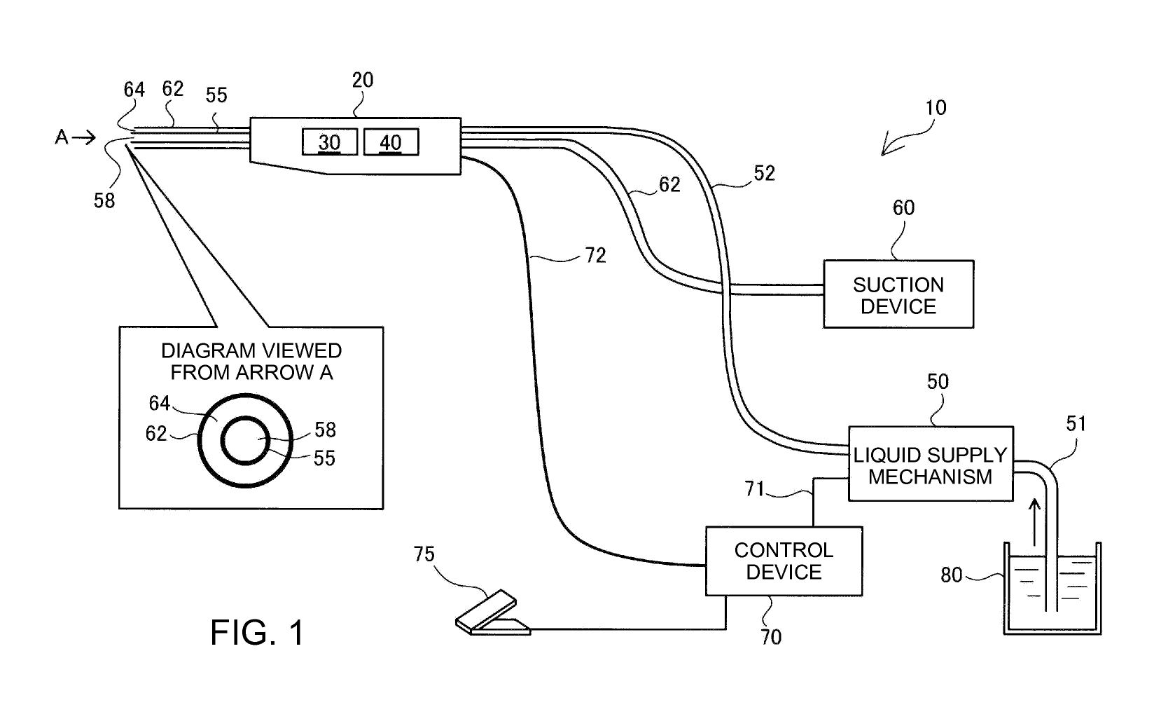

FIG. 1 shows the configuration of a liquid ejection apparatus 10. The liquid ejection apparatus 10 is medical equipment used in medical institutions, and has a function of incising or resecting the lesion by ejecting liquid to the lesion.

The liquid ejection apparatus 10 includes a liquid ejection mechanism 20, a liquid supply mechanism 50, a suction device 60, a control device 70, and a liquid container 80. The liquid supply mechanism 50 and the liquid container 80 are connected to each other through a connection tube 51. The liquid supply mechanism 50 and the liquid ejection mechanism 20 are connected to each other through a liquid supply passage 52. The connection tube 51 and the liquid supply passage 52 are formed of resin. The connection tube 51 and the liquid supply passage 52 may be formed of materials (for example, metal) other than resin.

The liquid container 80 contains a saline solution. Instead of the saline solution, pure water or a chemical solution maybe used. The liquid supply mechanism 50 supplies liquid, which is suctioned from the liquid container 80 through the connection tube 51, to the liquid ejection mechanism 20 through the liquid supply passage 52 by the driving of a built-in pump.

The liquid ejection mechanism 20 is a device that the user of the liquid ejection apparatus 10 operates while holding it in his or her hand. The user incises or resects the lesion by applying the liquid, which is intermittently ejected from the liquid ejection mechanism 20, to the lesion.

The liquid ejection mechanism 20 is a disposable product, and is replaced with a new product for each operation. In the present embodiment, the liquid ejection mechanism 20 (high power type liquid ejection mechanism 20) in which the excision capacity is set to be high and the liquid ejection mechanism 20 (low power type liquid ejection mechanism 20) in which the excision capacity is set to be low are prepared as new liquid ejection mechanisms 20. The user selects and prepares any of the liquid ejection mechanisms 20 according to an excision part or the like before the operation.

The liquid ejection mechanism 20 includes a storage unit 40. The storage unit 40 stores a liquid ejection mechanism ID (hereinafter, abbreviated as an “ID”). A unique ID is assigned to each liquid ejection mechanism 20. The ID includes information by which the high power type liquid ejection mechanism 20 or the low power type liquid ejection mechanism 20 can be determined.

The suction device 60 is used for suction of liquid or a resected part around an ejection port 58. The suction device 60 and the liquid ejection mechanism 20 are connected to each other through a suction passage 62. The suction device 60 suctions the inside of the suction passage 62 consistently while a switch for operating the suction device 60 is ON. The suction passage 62 passes through the inside of the liquid ejection mechanism 20 and is open in the vicinity of the tip of an ejection tube 55.

The suction passage 62 is covered by the ejection tube 55 extending from the tip of the liquid ejection mechanism 20. Therefore, as shown in a diagram viewed from the arrow A of FIG. 1, the wall of the ejection tube 55 and the wall of the suction passage 62 form approximately concentric cylinders. Between the outer wall of the ejection tube 55 and the inner wall of the suction passage 62, a passage through which a suctioned material, which is suctioned from a suction port 64 that is a tip of the suction passage 62, flows is formed. The suctioned material is suctioned into the suction device 60 through the suction passage 62.

The liquid supply passage 52, the suction passage 62, and a signal cable 72 (hereinafter, these three are referred to collectively as “cables”) are fixed to the liquid ejection mechanism 20, and are replaced together with the liquid ejection mechanism 20. When using the new liquid ejection mechanism 20, the liquid ejection mechanism 20 to which cables are connected is prepared, and the cables are connected to respective connection destinations.

When the user turns ON a foot switch 75 in a state where cables are connected, the control device 70 transmits a driving signal to a pulsation generating unit 30, which is built into the liquid ejection mechanism 20, through the signal cable 72. When the driving signal is input, the pulsation generating unit 30 generates a pulsation for the pressure of the supplied liquid. By this pulsation, intermittent ejection of the liquid described above is performed. The pulsation generating unit 30 generates the pulsation using the expansion and contraction of an actuator built therein. The actuator is configured by a piezoelectric element. The driving signal is for expanding and contracting the piezoelectric element.

Here, the ejection of liquid when the foot switch 75 is turned ON as described above occurs while the control device 70 is set to a permission mode. The control device 70 sets itself to either the permission mode or a non-permission mode. In the non-permission mode, even if the foot switch 75 is turned ON, the control device 70 does not drive the pulsation generating unit 30 and the liquid supply mechanism 50. Accordingly, in the non-permission mode, no liquid is ejected.

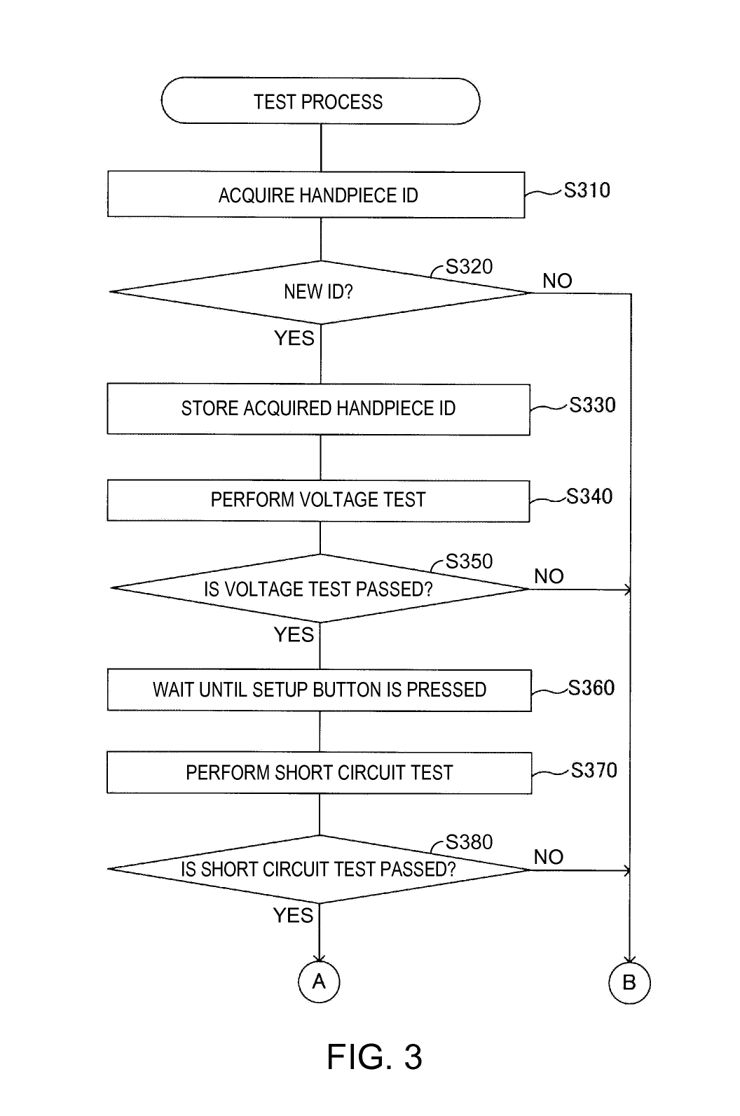

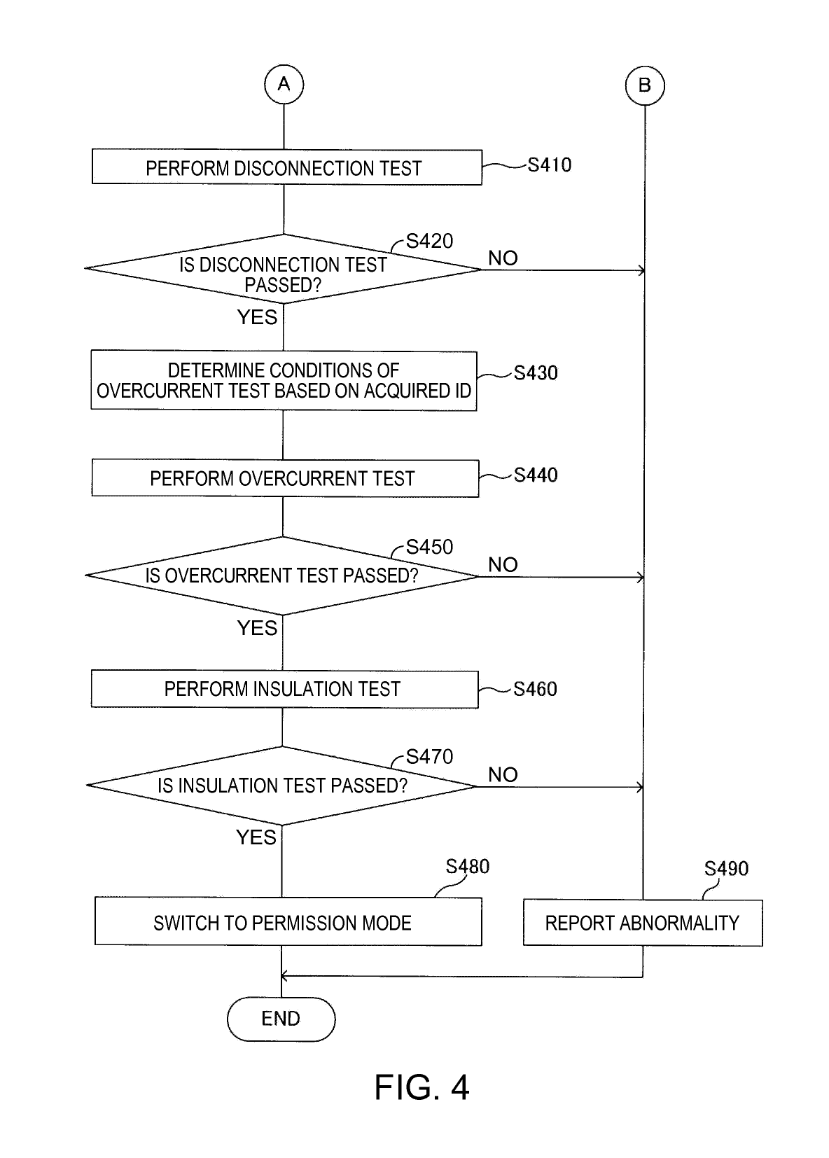

The default mode of the control device 70 is a non-permission mode. Switching to the permission mode is performed when a test process (which will be described later with reference to FIGS. 3 and 4) is performed after the connection of the signal cable 72 and the test is passed. The permission mode is maintained until the signal cable 72 is removed after the switching to the permission mode.

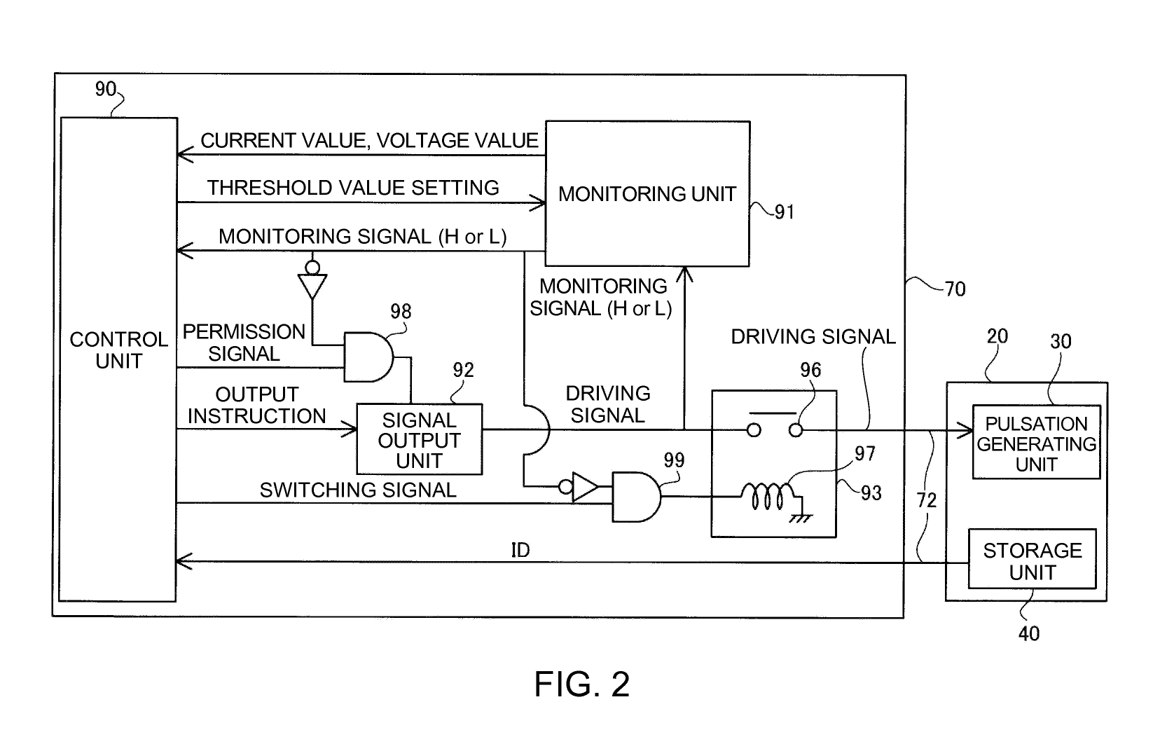

FIG. 2 is a block diagram showing the internal configuration of the control device 70, and shows a state in which the control device 70 and the liquid ejection mechanism 20 are connected to each other through the signal cable 72. The control device 70 includes a control unit 90, a monitoring unit 91, a signal output unit 92, a relay 93, a first AND circuit 98, and a second AND circuit 99. The relay 93 is an electromagnetic relay, and includes a contact point 96 and an actuating coil 97.

The control unit 90 is formed by a microcomputer, and includes a nonvolatile memory (for example, an FeRAM). The control unit 90 instructs the signal output unit 92 to output a driving signal. The signal output unit 92 outputs the driving signal when the instruction is received. The driving signal output from the signal output unit 92 is input to the monitoring unit 91 and the relay 93. In a state where the contact point 96 is closed (hereinafter, referred to as “when the relay 93 is ON”), the driving signal passes through the relay 93 and is then input to the pulsation generating unit 30 through the signal cable 72.

The monitoring unit 91 monitors the driving signal before being input to the relay 93. The monitoring unit 91 measures the voltage value and the current value of the driving signal, and inputs the measurement result to the control unit 90. The monitoring unit 91 outputs a value H, which indicates that each of the voltage value and the current value is equal to or greater than a set threshold value, and a value L, which indicates that each of the voltage value and the current value is less than the set threshold value. In FIG. 2, for convenience of illustration, digital signals for the voltage value and the current value are collectively shown as a “monitoring signal”. The threshold value described above is a variable value set by the control unit 90. The digital signal output from the monitoring unit 91 is input to the control unit 90, and is input to the first AND circuit 98 and the second AND circuit 99 after being inverted. This inversion is performed by an inverter element.

The control unit 90 performs switching between ON and OFF (state in which the contact point 96 is open) of the relay 93 by inputting a switching signal to the actuating coil 97 of the relay 93 through the second AND circuit 99. The contact point 96 is a normally open contact point. Accordingly, the relay 93 is ON when the switching signal is input, and is OFF when the switching signal is not input. The switching signal is input to the actuating coil 97 when the value L is input to the second AND circuit 99 as a monitoring signal for both the voltage value and the current value. That is, when a value equal to or greater than the threshold value, for at least one of the voltage value and the current value of the driving signal, is detected by the monitoring unit 91, the relay 93 is turned OFF to stop the driving signal.

The control unit 90 inputs a permission signal to the signal output unit 92 through the first AND circuit 98 when an output instruction is given to the signal output unit 92. Even if the output instruction is given, the signal output unit 92 does not output a signal unless a permission signal is input. The permission signal is input to the signal output unit 92 when the value L is output as a monitoring signal for both the voltage value and the current value. That is, when a value equal to or greater than the threshold value, for at least one of the voltage value and the current value of the driving signal, is detected by the monitoring unit 91, no driving signal is output.

When at least one of the voltage value and the current value input from the monitoring unit 91 is equal to or greater than a predetermined value, the control unit 90 stops the output of an output instruction, a permission signal, and a switching signal. If these outputs are stopped, no driving signal is input to the pulsation generating unit 30.

By the monitoring function of the control unit 90 and the monitoring unit 91 described above, a driving signal due to excessive voltage or current is not input to the pulsation generating unit 30.

It is preferable to check as often as possible whether or not the monitoring function works normally. In the present embodiment, this checking is performed as a test process, which will be described later, whenever the new liquid ejection mechanism 20 is used.

FIGS. 3 and 4 are flowcharts showing the test process. The test process is performed by the control unit 90 when the liquid ejection mechanism 20 is connected to the control device 70 through the signal cable 72. The control device 70 detects a connection to the liquid ejection mechanism 20 based on a change in the electric potential of the connection line of the signal cable 72 connected to the storage unit 40. The change in the electric potential is caused by a pull-up resistor and a pull-down resistor. As will be described later, when the test in this process is passed, the control device 70 proceeds to the permission mode from the non-permission mode.

First, an ID is acquired from the storage unit 40 (step S310). Then, it is determined whether or not the acquired ID is a new ID (step S320). Specifically, when the acquired ID does not match any ID stored in the control unit 90, it is determined that the acquired ID is a new ID. When the acquired ID matches one of the IDs stored in the control unit 90, it is determined that the acquired ID is not a new ID. The storage of the ID is performed in step S330 to be described later.

When the acquired ID is not a new ID (step S320; NO), it is reported that the liquid ejection mechanism 20 that has been connected to the control unit 90 before is connected (step S490), and the test process is ended. Here, the reporting of abnormalities is performed by outputting a message, such as “please replace the liquid ejection mechanism with a new one”. The output of the message is performed by display or voice. The output of the display or voice is performed by using a display or a speaker provided in the control device 70. Such a reporting using a message is performed because the acquired ID is not a new ID, and accordingly, it is estimated that the liquid ejection mechanism 20 is a used one. In this case, since a non-permission mode is maintained, the ejection of liquid by the liquid ejection mechanism 20 is not performed.

On the other hand, when the acquired ID is a new ID (step S320; YES), the acquired ID is stored in a storage medium (step S330). Then, a voltage test is performed (step S340). The voltage test is to test whether or not a voltage is generated from the signal output unit 92 according to the output instruction from the control unit 90 in a state where OFF of the relay 93 is maintained. Whether or not a voltage is generated according to the output instruction is determined by comparing the output instruction given to the signal output unit 92 with a voltage value input from the monitoring unit 91.

When the voltage test is not passed (step S350; NO), the above-described step S490 is performed. In this case, failure of the voltage, necessity of repair, or the like is reported. Even if an excessive voltage is generated, the application of the voltage to the pulsation generating unit 30 is avoided since the relay 93 is set to OFF.

On the other hand, when the voltage test is passed (step S350; YES), the control unit 90 waits until a setup button is pressed (step S360). The setup button is an input interface provided in the control device 70, and the user is requested to press the setup button after connecting the liquid ejection mechanism 20. Since a subsequent test is performed by turning ON the relay 93, a voltage is applied to the pulsation generating unit 30. Therefore, in order to call a user's attention, pressing of the setup button is requested. In addition, since the liquid supply mechanism 50 is not driven in the test process, no liquid is ejected from the liquid ejection mechanism 20.

After the setup button is pressed, a short circuit test is performed (step S370). The short circuit test is a test for checking whether or not a short circuit has occurred in the connected liquid ejection mechanism 20.

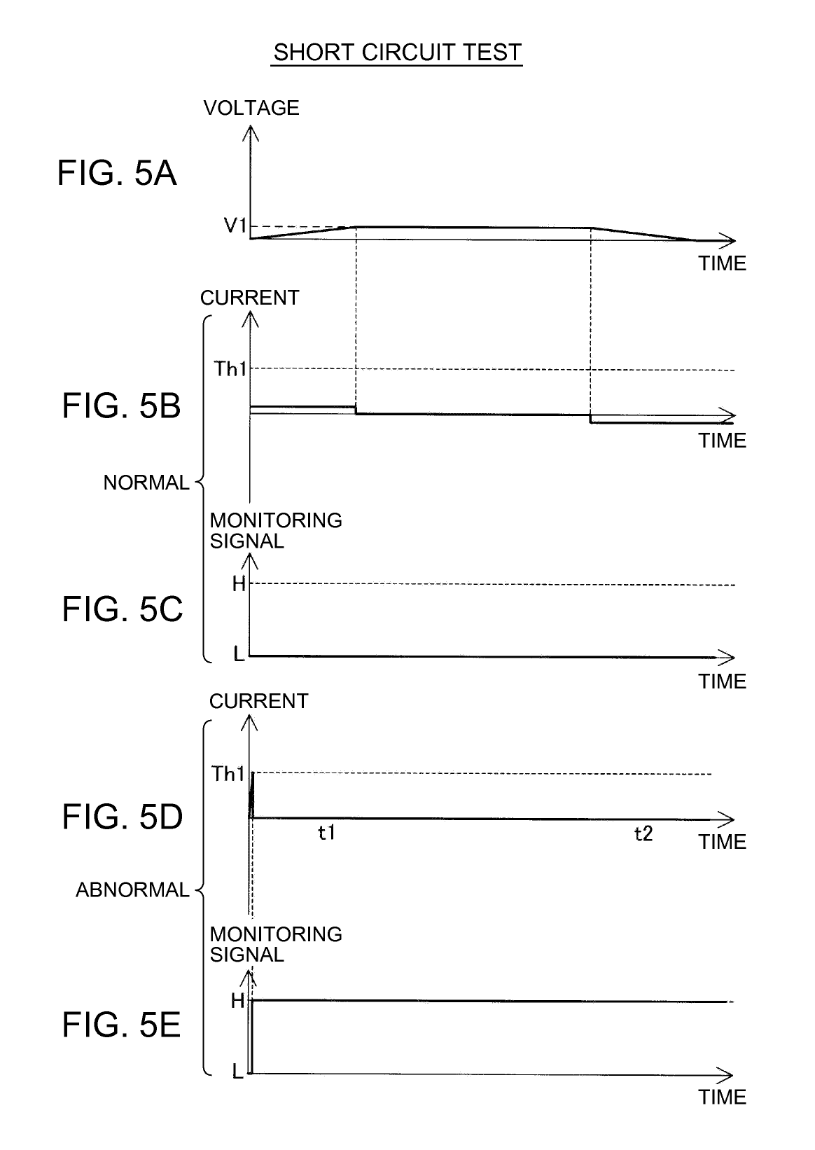

FIGS. 5A to 5E are graphs showing various waveforms in the short circuit test. FIG. 5A shows a temporal change of the voltage of a short circuit test signal. FIG. 5B shows a temporal change of the current in the normal state. The normal state referred to herein means that no short circuit occurs in the signal cable 72 and the like. FIG. 5C shows a monitoring signal as a current monitoring result in the normal state. FIG. 5D shows a temporal change of the current when a short circuit occurs. FIG. 5E shows a monitoring signal as a current monitoring result when a short circuit occurs.

As shown in FIG. 5A, the waveform of the short circuit test signal is a trapezoidal shape. That is, the voltage of the short circuit test signal rises linearly up to a voltage V1, and the voltage V1 is maintained for a predetermined amount of time. After the predetermined amount of time has passed, the voltage of the short circuit test signal drops linearly until the voltage becomes 0. The voltage V1 is set to a voltage much lower than the maximum voltage of the driving signal (for example, 1/10 or less of the maximum voltage of the driving signal) in consideration of a possibility of a short circuit. By setting the voltage V1 to a voltage much lower than the maximum voltage of the driving signal, it is possible to suppress the damage to the electrical circuit or the malfunction of the electrical circuit even if a short circuit occurs.

The short circuit test signal is input to the piezoelectric element. As shown in FIG. 5B, a positive current flows during a period for which the voltage rises linearly, no current flows during a period for which the voltage is maintained at the voltage V1, and a negative current flows during a period for which the voltage drops linearly. Being maintained at the voltage V1 means falling within the range of a predetermined voltage value.

In the short circuit test, a threshold value Th1 is set for the current value. The monitoring unit 91 outputs the value L when the current value is maintained at a value less than the threshold value Th1, and outputs the value H when the current value reaches the threshold value Th1.

As shown in FIG. 5B, the threshold value Th1 corresponds to a value of current that does not flow at the voltage V1 in the normal state. Therefore, in the normal state, the monitoring signal is maintained at the value L. When the monitoring signal is maintained at the value L, the control unit 90 determines that the state is normal since a short circuit has not occurred.

On the other hand, when a short circuit occurs, as shown in FIG. 5D, the current value reaches the threshold value Th1 immediately after the input of the short circuit test signal. When the current value reaches the threshold value Th1, a protection function of the control unit 90 and the monitoring unit 91 operates as described above. Therefore, as shown in FIG. 5D, the current value becomes 0 after reaching the threshold value Th1. The control unit 90 determines that a short circuit has occurred when the monitoring signal reaches the value H. The threshold value for the voltage value is set to a value larger than the maximum voltage so as not to interfere with the determination based on the current value. This is also the same for all subsequent tests.

The short circuit test is performed as described above, and the above-described step S490 is performed when the test is not passed (step S380; NO). In this case, a message, such as “Abnormality has been detected in the liquid ejection mechanism. Please replace it”, is output.

When the short circuit test is passed (step S380; YES), a disconnection test is performed (step S410). The disconnection test is a test for checking whether or not disconnection has occurred in the signal cable 72 or the like.

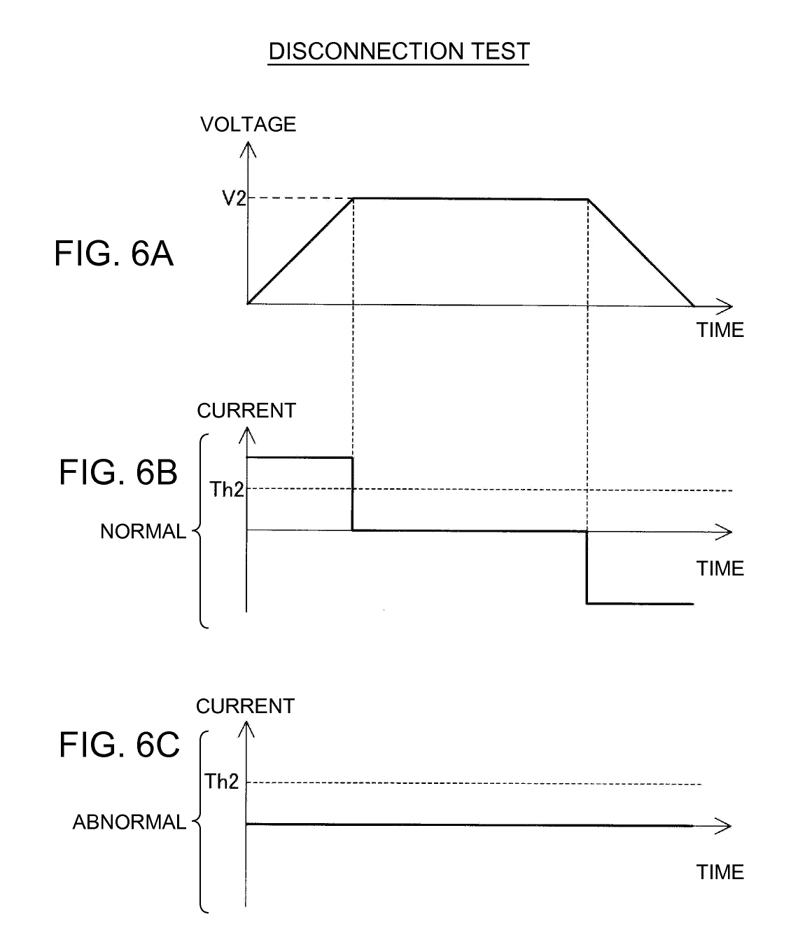

FIGS. 6A to 6C are graphs showing various waveforms in the disconnection test. FIG. 6A shows a temporal change of the voltage of a disconnection test signal. FIG. 6B shows a temporal change of the current in the normal state. The normal state referred to herein means that no disconnection occurs in the signal cable 72 and the like. FIG. 6C shows a temporal change of the current when disconnection occurs.

As shown in FIG. 6A, the waveform of the disconnection test signal is a trapezoidal shape in the same manner as the short circuit test signal, and the maximum voltage is a voltage V2. The voltage V2 is higher than the voltage V1 that is the maximum voltage of the short circuit test signal, and is lower than the maximum voltage of the driving signal.

As shown in FIG. 6B, the waveform of the current value in the normal state is stepwise as in the case of the short circuit test. When the current value is equal to or greater than the threshold value Th2, the control unit 90 determines that the state is normal since disconnection has not occurred. The threshold value Th2 corresponds to a current value lower than a value of current that flows at the voltage V2 if disconnection does not occur.

The threshold value Th2 is not a threshold value set in the monitoring unit 91 but is a value that the control unit 90 adopts as criteria. This is because, if the threshold value Th2 is set in the monitoring unit 91, the current value becomes 0 immediately after the start of a test, and accordingly, it is difficult to determine whether or not the state is normal. The threshold value set in the monitoring unit 91 in the disconnection test is set to a larger value than the current value generated in the disconnection test.

On the other hand, the current value when disconnection occurs is maintained at 0, as shown in FIG. 6C. Thus, when the current value does not reach the threshold value Th2, the control unit 90 determines that disconnection has occurred.

When the disconnection test is not passed (step S420; NO), the above-described step S490 is performed. Also in this case, failure of the wiring system, necessity of repair, or the like is reported.

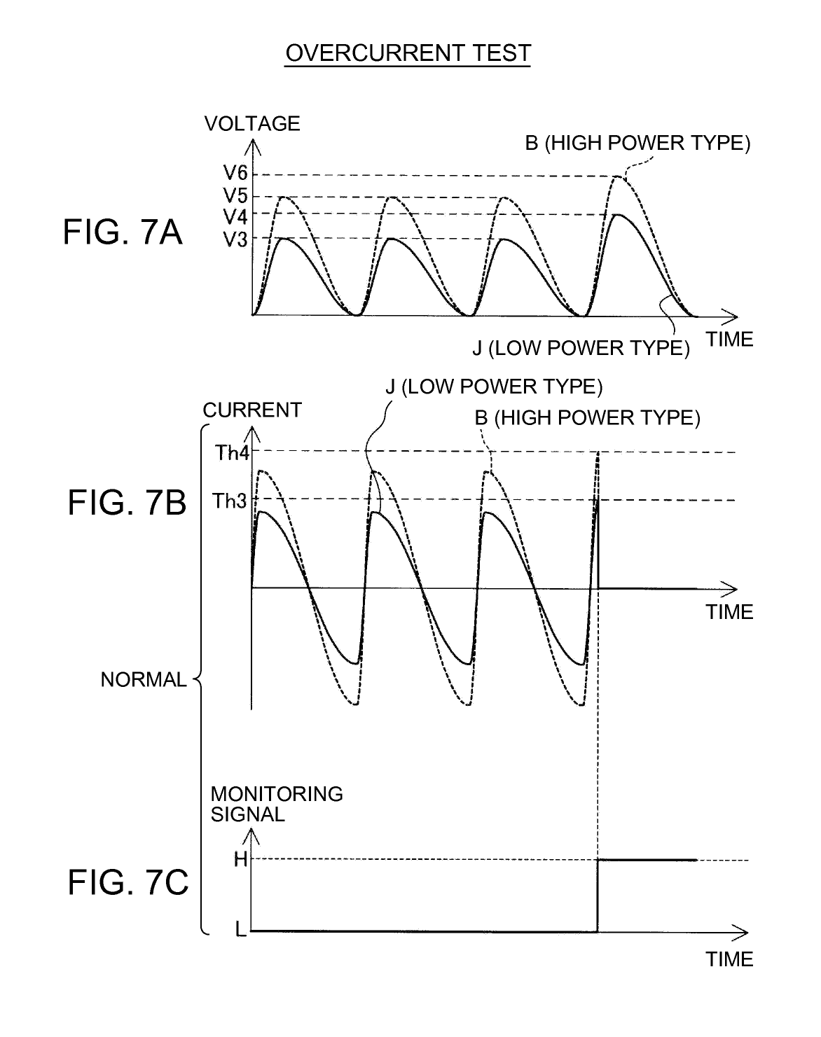

When the disconnection test is passed (step S420; YES), the test conditions of an overcurrent test are determined based on the acquired ID (step S430), and the overcurrent test is performed (step S440). The overcurrent test is a test for checking whether or not the protection function described above operates normally when a current equal to or higher than the set threshold value is generated. The overcurrent test and the test conditions will be described with reference to FIGS. 7A to 7C.

FIGS. 7A to 7C are graphs showing various waveforms in the overcurrent test. FIG. 7A shows a temporal change of the voltage in an overcurrent test signal. FIG. 7B shows a temporal change of the current in the overcurrent test signal. FIG. 7C shows a temporal change of the monitoring signal in the overcurrent test. A solid line J in FIGS. 7A and 7B indicates a case of the low power type liquid ejection mechanism 20, and a broken line B indicates a case of the high power type liquid ejection mechanism 20.

In the case of the low power type liquid ejection mechanism 20, a current equal to or higher than a threshold value Th3 shown in FIG. 7B is regarded as an overcurrent, and the threshold value Th3 is set in the monitoring unit 91. Then, as shown in FIG. 7A, a driving signal having a voltage V3 as a maximum voltage is output as the overcurrent test signal for a predetermined amount of time. The voltage V3 is five times or more than the voltage V1. The driving signal is a driving signal output in the use mode, and does not generate a current equal to or greater than the threshold value Th3 as shown in

FIG. 7B. The predetermined amount of time is an arbitrary time, and is illustrated as three periods of the driving signal in FIG. 7A. Preferably, the voltage V3 is 10 times or more than the voltage V1. When the voltage V3 is 10 times or more than the voltage V1, the test can be performed more accurately.

A driving signal having the voltage V3 as a maximum voltage is output for a predetermined amount of time, and then a driving signal having a voltage V4 as a maximum voltage is output. The voltage V4 is a voltage value for generating a current equal to or greater than the threshold value Th3. When the current equal to or greater than the set threshold value is generated, the value H is output as a monitoring signal, as shown in FIG. 7C. As described previously in the short circuit test, when the value H is output, the protection function of the control unit 90 and the monitoring unit 91 operates, and the current value becomes 0 as shown in FIG. 7B. The control unit 90 determines that the overcurrent test has been passed when the current becomes 0 as described above, and determines that the overcurrent test has not been passed when the current does not become 0.

The test conditions in the case of the low power type liquid ejection mechanism 20 include the threshold value Th3, the voltage V3, and the voltage V4 described above. In the case of the high power type liquid ejection mechanism 20, as shown in FIGS. 7A and 7B, a threshold value Th4 (>threshold value Th3), a voltage V5 (>voltage V3), and a voltage V6 (>voltage V4) are adopted instead of the threshold value Th3, the voltage V3, and the voltage V4. The reason why the conditions are changed as described above is that, in the case of the high power type liquid ejection mechanism 20, the maximum voltage of the driving signal is high, and accordingly, the current value regarded as the overcurrent is large.

In addition, when the protection function operates as described above, the overcurrent test signal is interrupted and the voltage becomes 0. In FIG. 7A, however, for convenience of explanation and description, the overcurrent test signal is output even after the protection function operates.

When the overcurrent test is not passed (step S450; NO), the above-described step S490 is performed. In this case, since a possibility of the failure of the control device 70 is high, failure of the control device, the necessity of repair, or the like is reported.

When the overcurrent test is passed (step S450; YES), an insulation test is performed (step S460). The insulation test is a test for checking whether or not the current is held at 0 when a voltage applied to the pulsation generating unit 30 is fixed, that is, when there is no AC component in the voltage.

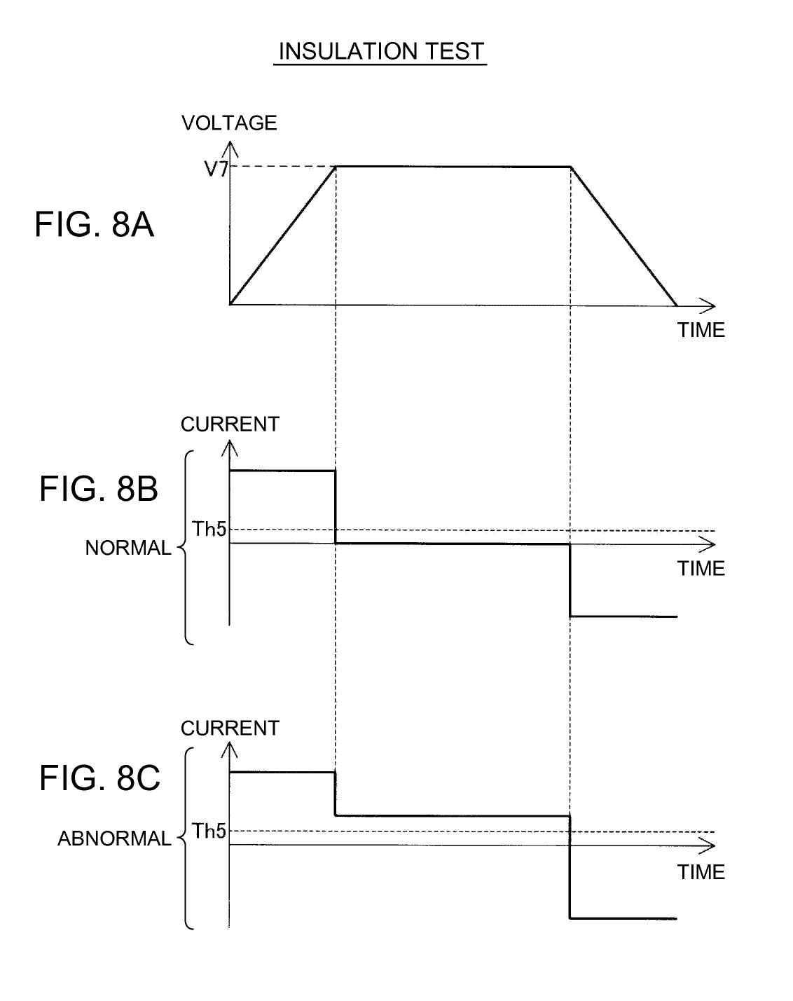

FIGS. 8A to 8C are graphs showing various waveforms in the insulation test. FIG. 8A shows a temporal change of the voltage of an insulation test signal. FIG. 8B shows a temporal change of the current in the normal state. The normal state referred to herein means that insulation is successfully made. FIG. 8C shows a temporal change of the current when there is no insulation.

As shown in FIG. 8A, the waveform of the insulation test signal is a trapezoidal shape in the same manner as the short circuit test signal and the disconnection test signal, and the maximum voltage is a voltage V7. The voltage V7 is set to a value higher than the voltages V1 to V6 in order to test insulation.

If the insulation is successfully made, no current flows while the voltage is held at the voltage V7, as shown in FIG. 8B. On the other hand, if there is no insulation, a current flows while the voltage is held at the voltage V7, as shown in FIG. 8C. When the current value equal to or greater than a threshold value Th5 is not detected, the control unit determines that the insulation is successfully made. Similar to the threshold value Th2 in the disconnection test, the threshold value Th5 is a value adopted as criteria in the control unit 90.

When the insulation test is not passed (step S470; NO), the above-described step S490 is performed. In this case, since the failure of the piezoelectric element is estimated as a cause of poor insulation, a message, such as “Abnormality has been detected in the liquid ejection mechanism. Please replace it”, is output.

When the insulation test is passed (step S470; YES), switching to the permission mode is performed (step S480), and the test process is ended. After the switching to the permission mode, liquid is ejected from the liquid ejection mechanism 20 by turning on the foot switch 75.

According to the present embodiment, various tests of the liquid ejection mechanism 20 and the control device 70 can be performed before the operation. By the tests, it is possible to prevent the used liquid ejection mechanism 20 from being reused or to prevent the operation from being performed in a state where there is an abnormality. When an abnormality is detected, it is possible to prompt the user to perform replacement or repair. In addition, in the overcurrent test, it is possible to perform a test according to the output type of the liquid ejection mechanism 20.

The short circuit test in the embodiment corresponds to a first test step in the aspects of the invention. The overcurrent test in the embodiment corresponds to a second test step in the aspects of the invention. The voltage V1 corresponds to a first voltage, and the voltages V5 and V6 correspond to a second voltage.

In the present embodiment, whenever the liquid ejection mechanism 20 that has never been used is connected to the control device 70, anyone of the voltage test, the short circuit test, the disconnection test, the overcurrent test, and the insulation test may be performed. Thus, safe and reliable medical equipment can be provided for each operation.

In addition, the control device 70 may perform the voltage test when a predetermined amount of time has passed, and any one of the voltage test, the short circuit test, the disconnection test, the overcurrent test, and the insulation test may be performed whenever the liquid ejection mechanism 20 that has never been used is connected to the control device 70. For example, the control device 70 includes a timer, and performs a voltage test when the liquid ejection mechanism 20 that has never been used is connected to the control device 70 after 24 hours since the use. In this case, even if a plurality of liquid ejection mechanisms 20 are connected to the control device 70 within 24 hours, the liquid ejection apparatus can be used earlier since the voltage test can be omitted.

The invention is not limited to the embodiments, examples, or modification examples of this specification, and various configurations can be implemented without departing from the spirit and scope of the invention. For example, in order to solve some or all of the problems described above or to achieve some or all of the effects described in this specification, technical features in the embodiments, examples, and modification examples corresponding to the technical features described in the aspects of the invention may be appropriately replaced or combined. The technical features can be appropriately deleted if the technical features are not described as essential ones. For example, the following may be mentioned.

Although the ID is acquired from the storage unit 40 and it is determined whether or not the acquired ID is a new ID (step S320), the invention is not limited thereto. Specifically, an ID allowing the connection to the control unit is stored in advance, and the control unit 90 stores connection history when the liquid ejection mechanism 20 is connected to the control unit 90. Then, when the liquid ejection mechanism 20 that has been connected before is connected again, the test process may be ended, and a test of the liquid ejection mechanism 20 that has never been connected may be performed.

Instead of the connection history, a period for which the control unit 90 uses the liquid ejection mechanism 20 or a period for which the liquid ejection mechanism 20 is connected to the control unit 90 may be stored, or one or more of the connection history, the use period, and the connection period may be stored. Specifically, when the liquid ejection mechanism 20 is connected to the control unit 90, the control unit 90 stores one or more of the connection history, the use period, and the connection period. Then, one or more items regarding whether or not there is connection history, whether or not a predetermined use period has expired, and whether or not a predetermined connection period has expired may be checked. When the liquid ejection mechanism. 20 corresponding to one or more of the items is connected again, the test process may be ended, and the liquid ejection mechanism 20 that does not correspond to one or more of the items may be tested. By storing the use period or the connection period of the liquid ejection mechanism 20, the voltage test, the short circuit test, the disconnection test, the overcurrent test, and the insulation test can be performed even if the liquid ejection mechanism 20 is attached and detached multiple times in one operation.

By performing the voltage test, the short circuit test, the disconnection test, the overcurrent test, and the insulation test whenever the liquid ejection mechanism 20 is connected or at predetermined time intervals, it is possible to provide safe and reliable medical equipment. As a test of the electrical system of the liquid ejection mechanism 20, a current test maybe performed in addition to the voltage test, the short circuit test, the disconnection test, the overcurrent test, and the insulation test. The current test may also be performed instead of the voltage test.

At least two of the voltage test, the short circuit test, the disconnection test, the overcurrent test, and the insulation test, for example, the disconnection test and the insulation test may be performed using the same test signal.

It is preferable to perform the disconnection test, the overcurrent test, and the insulation test after performing the voltage test and the short circuit test. In this case, the voltage test and the short circuit test may be performed in any order, and the disconnection test, the overcurrent test, and the insulation test may be performed in any order.

Some of the voltage test, the short circuit test, the disconnection test, the overcurrent test, and the insulation test may not be performed.

When the short circuit test is not performed, the disconnection test may be performed as a first test step.

The insulation test maybe performed as a second test step. In this case, the voltage used in the insulation test may be changed according to the type of the liquid ejection mechanism.

A test corresponding to the second test step may be performed under the same conditions regardless of an ID.

ID acquisition may be performed at any time before the test corresponding to the second test step. For example, the ID acquisition may be performed after the short circuit test or may be performed after the disconnection test.

The magnitude relationship of the voltages in the test signals shown in the embodiment is just an example, and may be changed.

The waveform of the signal used in each test may be changed. For example, the waveform of the signal used in each test may be changed to a triangular wave.

The success/failure determination in each test is not limited to that illustrated in the embodiment, but various determinations may be considered. For example, in the overcurrent test, success or failure may be determined based on the fact that the control unit can detect the overcurrent successfully even if the current is not actually interrupted.

The liquid ejection mechanism and the cables may not be fixed. For example, the cables may be fixed to the control device, the liquid supply mechanism, and the suction device.

There maybe three or more output types of the liquid ejection mechanism.

An identifier may be used to identify the liquid ejection mechanism described in the embodiment and other liquid ejection mechanisms.

As other liquid ejection mechanisms, it is possible to use a liquid ejection mechanism that is used for an endoscope, such as a laparoscope, and is inserted into the body and is operated.

The liquid ejection apparatus may be used for apparatuses other than the medical equipment.

For example, the liquid ejection apparatus may be used for a cleaning apparatus that removes dirt with the ejected liquid.

The liquid ejection apparatus may be used for a drawing apparatus that draws a line or the like with the ejected liquid.

As a liquid ejection method, laser light may be used. As an ejection method using the laser light, for example, it is possible to apply a method using a pressure variation due to the evaporation of the liquid caused when emitting the laser light intermittently to the liquid.

What is claimed is:

1. A control device that is connectable to a surgical instrument and controls the surgical instrument,
wherein, when the surgical instrument is connected, a quality of the surgical instrument is determined by performing a plurality of different tests.

2. The control device according to claim 1,
wherein the surgical instrument has an identifier, the control device includes
a test unit that applies a voltage to the surgical instrument, and
an identification unit that identifies the identifier, and
the test unit changes the voltage according to the identifier.

3. The control device according to claim 1,
wherein the surgical instrument has an identifier,
the control device includes
a first test unit that applies a first voltage to the surgical instrument,
an identification unit that identifies the identifier, and
a second test unit that applies a second voltage higher than the first voltage to the surgical instrument, and
the second test unit operates after operations of the first test unit and the identification unit.

4. A control device that is connectable to a liquid ejection mechanism having an identifier and outputs a control signal for controlling the liquid ejection mechanism, comprising:
a first test unit that applies a first voltage to the liquid ejection mechanism;
an identification unit that identifies the identifier; and
a second test unit that applies a second voltage higher than the first voltage to the liquid ejection mechanism,
wherein the second test unit operates after operations of the first test unit and the identification unit.

5. The control device according to claim 4,
wherein the liquid ejection mechanism is connectable, and
the identification unit operates when the liquid ejection mechanism is connected.

6. A test method of medical equipment including a surgical instrument having an identifier and a control device that outputs a driving signal to the surgical instrument, the method comprising:
applying a first voltage to the surgical instrument;
identifying the identifier; and
applying a second voltage higher than the first voltage to the surgical instrument,
wherein the application of the second voltage is performed after the application of the first voltage and the identification of the identifier.

7. The test method according to claim 6,
wherein the identifier has different information for each type of the surgical instrument.

8. The test method according to claim 7,
wherein a value of the second voltage is changed according to the identifier.

9. The test method according to claim 6,
wherein a value of the second voltage is five times or more than a value of the first voltage.

10. The test method according to claim 6,
wherein the identification of the identifier is performed before the application of the first voltage.

11. The test method according to claim 6,
wherein the application of the first voltage is performed before the identification of the identifier.

12. The test method according to claim 6,
wherein the identifier has information unique to each surgical instrument.

13. The test method according to claim 12,
wherein, when an identifier acquired in the identification of the identifier is the same as an identifier acquired in the previous identification of the identifier, the application of the second voltage is not performed.

14. The test method according to claim 6,
wherein the surgical instrument includes an actuator.

15. The test method according to claim 6,
wherein the medical equipment is a liquid ejection mechanism.
